(19) United States
(12) Patent Application Publication
(10) Pub. No.: US 2024/0050005 A1
(43) Pub. Date: Feb. 15, 2024

(54) COMMUNICATION APPARATUS, COMMUNICATION METHOD, AND NON-TRANSITORY COMPUTERREADABLE STORAGE MEDIUM

(71) Applicant: NEC Corporation, Tokyo (JP)

(72) Inventors: Keiju NAKAMURA, Tokyo (JP); Hiroaki KISO, Tokyo (JP)

(73) Assignee: NEC Corporation, Tokyo (JP)

(21) Appl. No.: 18/229,473

(22) Filed: Aug. 2, 2023

(30) Foreign Application Priority Data
Aug. 12, 2022 (JP) 2022-129012

(51) Int. Cl. A61B 5/16; G06V 40/16; A61B 5/00; G16H 20/10

(52) U.S. Cl. CPC A61B 5/165; G06V 40/174; A61B 5/4848; G16H 20/10

(57) ABSTRACT

A communication apparatus includes a processing unit, a judgement unit, and a communication unit. The processing unit generates state information indicating a state of a target person by processing an image in which the target person is captured. The judgement unit performs, by using the state information, at least one of processing of determining first information needed to be transmitted to a first terminal and processing of deciding whether to transmit the first information to the first terminal. The first terminal is associated with the target person, and is operated by a different person from the target person. The communication unit transmits the first information to the first terminal. The communication apparatus can also support decision-making on how to respond to the target person.

This application is based upon and claims the benefit of priority from Japanese Patent Application No. 2022-129012 filed on Aug. 12, 2022, the disclosure of which is incorporated herein in its entirety by reference.

### TECHNICAL FIELD

The present invention relates to a communication apparatus, a communication method, and a program.

### BACKGROUND ART

In recent years, it has been considered to analyze a state of a person and use the analysis result when a service is provided. For example, Patent Document 1 (Japanese Patent Application Publication No. 2015-018301) discloses that a feeling of a customer is used when a potential need of the customer is visualized. Specifically, a sales support terminal acquires information that determines a product from information about the product input via input equipment. Next, the sales support terminal acquires information representing a feeling of a customer about the product from the information about the product, and generates information in which the information that determines the product is associated with the information representing the feeling of the customer about the product. Then, the sales support terminal generates and outputs information that supports sales, based on the associated information.

Note that, Patent Document 2 (Japanese Patent Application Publication No. 2017-167985) discloses an apparatus for a purpose of connecting a product that interests a customer in a waiting time at a window to sales at the window. Specifically, the apparatus acquires data for tracking a customer at first timing at which a reception number is issued by a reception machine that receives waiting for a turn at a window. Next, the apparatus detects a product that satisfies a predetermined interest detection condition. Next, when the data for tracking the customer to be acquired at second timing at which the product is detected is similar to the data for tracking the customer being acquired at the first timing, the apparatus associates the reception number issued at the first timing with the product detected at the second timing. Next, when the reception number in which the waiting for a turn at a window is received is assigned to an operator terminal used by an operator at the window, the apparatus notifies, of the product associated with the reception number, the operator terminal assigned with the reception number.

### SUMMARY OF INVENTION

Both of Patent Documents 1 and 2 described above do not consider communication between a plurality of persons.

One example of an object of the present invention is to provide a communication apparatus, a communication method, and a program that support communication between a plurality of persons.

One aspect of the present invention provides a communication apparatus including:
at least one memory configured to store instructions; and
at least one processor configured to execute the instructions to:
generate state information indicating a state of a target person by processing an image in which the target person is captured;
perform, by using the state information, at least one of processing of determining first information needed to be transmitted to a first terminal being associated with the target person and being operated by a different person from the target person and processing of deciding whether to transmit the first information to the first terminal; and
transmit the first information to the first terminal.

One aspect of the present invention provides a communication method including,
by at least one computer:
generating state information indicating a state of a target person by processing an image in which the target person is captured;
performing, by using the state information, at least one of processing of determining first information needed to be transmitted to a first terminal being associated with the target person and being operated by a different person from the target person and processing of deciding whether to transmit the first information to the first terminal; and
transmitting the first information to the first terminal.

One aspect of the present invention provides a program causing a computer to execute:
a process of generating state information indicating a state of a target person by processing an image in which the target person is captured;
a process of performing, by using the state information, at least one of processing of determining first information needed to be transmitted to a first terminal being associated with the target person and being operated by a different person from the target person and processing of deciding whether to transmit the first information to the first terminal; and
a process of transmitting the first information to the first terminal.

According to one aspect of the present invention, a state of a person can be used when communication between a plurality of persons is supported.

### BRIEF DESCRIPTION OF THE DRAWINGS

FIG. 1 is a diagram illustrating a main part of a communication apparatus according to an example embodiment.

FIG. 2 is a diagram illustrating one example of a usage environment of the communication apparatus.

FIG. 3 is a diagram illustrating one example of a functional configuration of a first terminal.

FIG. 4 is a diagram illustrating one example of a functional configuration of the communication apparatus.

FIG. 5 is a diagram illustrating one example of information stored in a storage unit.

FIG. 6 is a diagram illustrating a hardware configuration example of the communication apparatus.

FIG. 7 is a flowchart illustrating an operation performed by the communication apparatus together with an operation performed by the first terminal and a second terminal.

FIG. 8 is a diagram illustrating a modification example of FIG. 7.

FIG. 9 is a flowchart illustrating a first specific example of step S50.

FIG. 10 is a flowchart illustrating a second specific example of step S50.

FIG. 11 is a flowchart illustrating a third specific example of step S50.

### EXAMPLE EMBODIMENT

Hereinafter, example embodiments of the present invention will be described with reference to the drawings. Note that, in all of the drawings, a similar component has a similar reference sign, and description thereof will be appropriately not included.

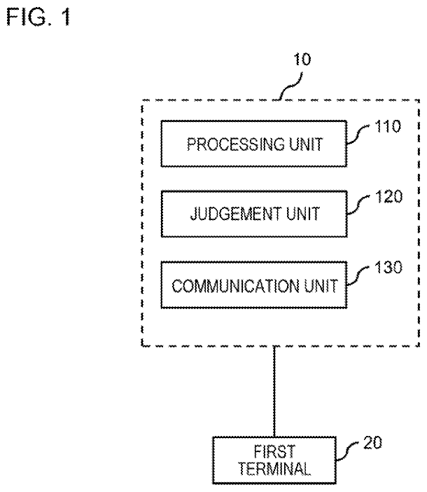

FIG. 1 is a diagram illustrating a main part of a communication apparatus 10 according to an example embodiment. The communication apparatus 10 includes a processing unit 110, a judgement unit 120, and a communication unit 130. The processing unit 110 generates state information indicating a state of a target person by processing an image in which the target person is captured. The judgement unit 120 performs, by using the state information, at least one of processing of determining first information needed to be transmitted to a first terminal 20 and processing of deciding whether to transmit the first information to the first terminal 20. The first terminal 20 is associated with the target person, and is operated by a different person from the target person. The communication unit 130 transmits the first information to the first terminal 20.

In this way, the judgement unit 120 performs, by using state information, at least one of processing of determining first information needed to be transmitted to the first terminal 20 and processing of deciding whether to transmit the first information to the first terminal 20. Therefore, the communication apparatus 10 can use a state of a target person when communication between a plurality of persons is supported.

One example of the person who operates the first terminal 20 is a relative of the target person.

For example, when the target person is a resident in a facility such as a care facility and a hospital, it is difficult for a relative of this resident to readily communicate with the resident. In contrast, when the communication apparatus 10 is used, the relative can easily communicate with the resident. Further, according to a state, for example, a mental state of the resident, the communication apparatus 10 determines first information, and decides whether to transmit the first information to the first terminal 20. Therefore, the relative can easily recognize a state of the resident.

Details of the communication apparatus 10 will be described below.

In the example illustrated in FIG. 1, the communication apparatus 10 is used together with the first terminal 20. The first terminal 20 is operated by a person other than a target person. Meanwhile, the communication apparatus 10 is operated by the target person or an assistant. In other words, the communication apparatus 10 assists in communication between the target person and the person other than the target person by communicating with the first terminal 20. For example, when the target person is a resident in a facility, the first terminal 20 is operated by a relative of the resident, and the communication apparatus 10 is operated by the resident, a caregiver, or a nurse. Further, when the target person is a relative of a resident in a facility, the first terminal 20 is operated by the resident, a caregiver, or a nurse, and the communication apparatus 10 is operated by the relative of the resident.

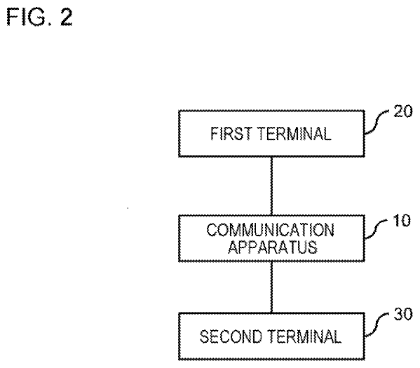

FIG. 2 is a diagram illustrating a second example of a usage environment of the communication apparatus 10. In the example illustrated in FIG. 2, the communication apparatus 10 is used together with a second terminal 30 in addition to the first terminal 20. In this case, one of the communication apparatus 10 and the second terminal 30 is operated by a caregiver or a nurse, and the other is operated by a target person.

Note that, in the example illustrated in FIG. 2, the communication apparatus 10 may be a terminal of a stationary type operated by, for example, a caregiver or a nurse, or may be a cloud server.

Further, in the examples in FIGS. 1 and 2, a cloud server may be further used in addition to the communication apparatus 10, the first terminal 20, and the second terminal 30. In this case, at least a part of processing performed by any of the communication apparatus 10, the first terminal 20, and the second terminal 30 may be performed by the cloud server. When the cloud server is used, the communication apparatus 10 is used together with a terminal being an interface of this cloud server in addition to the cloud server.

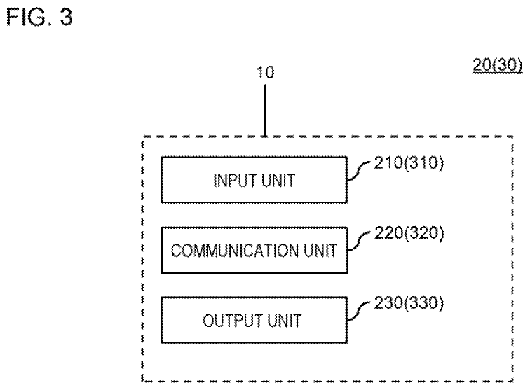

FIG. 3 is a diagram illustrating one example of a functional configuration of the first terminal 20. The first terminal 20 includes an input unit 210, a communication unit 220, and an output unit 230. One example of the first terminal 20 is a portable terminal such as a smartphone and a tablet type terminal.

The input unit 210 also functions as a capturing unit, and various types of information are input. For example, the input unit 210 includes a capturing apparatus, and generates an image. The image may be a moving image, may be a still image, or may include both of a moving image and a still image. Further, the input unit 210 may generate sound data together with an image. Further, text data may be input from a user to the input unit 210.

The communication unit 220 transmits and receives information to and from an external apparatus. One example of the external apparatus is the communication apparatus 10.

The output unit 230 includes an output apparatus, and outputs information by using this output apparatus. One example of this output apparatus is a display such as a touch panel, and a speaker. Note that, when the output unit 230 includes a touch panel, the output unit 230 also functions as an input apparatus operated by a user of the first terminal 20.

A functional configuration of the second terminal 30 illustrated in FIG. 2 is also similar to the functional configuration of the first terminal 20. In other words, the second terminal 30 includes an input unit 310, a communication unit 320, and an output unit 330. The input unit 310 is similar to the input unit 210, the communication unit 320 is similar to the communication unit 220, and the output unit 330 is similar to the output unit 230.

Note that, the first terminal 20 and the second terminal 30 may have another configuration. For example, when iris information, fingerprint information, or vein information is used at a time of authentication by the communication apparatus 10, the first terminal 20 and the second terminal 30 may include an apparatus for acquiring the information. Then, the information acquired by this apparatus is transmitted from the output unit 230 or the output unit 330 to the communication apparatus 10.

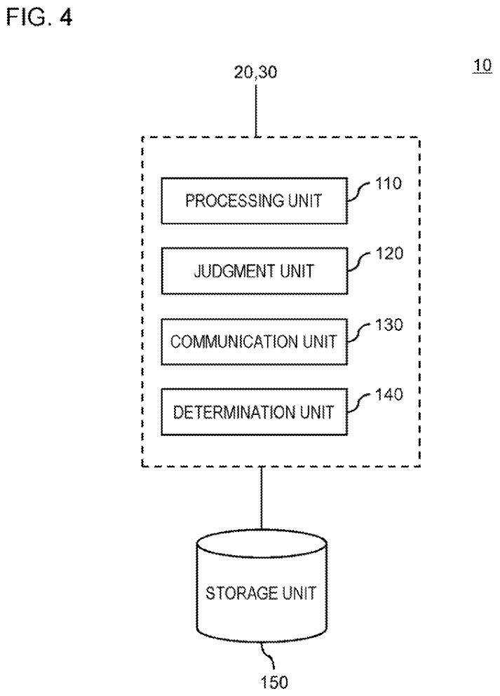

FIG. 4 is a diagram illustrating one example of a functional configuration of the communication apparatus 10. The communication apparatus 10 includes the processing unit 110, the judgement unit 120, the communication unit 130, and a determination unit 140, and can use a storage unit 150. In the example illustrated in FIG. 4, the storage unit 150 is a part of the communication apparatus 10, but may be located outside the communication apparatus 10.

The processing unit 110 authenticates a target person by processing an image including a face of the target person. The processing unit 110 acquires this image from the second terminal 30 via the communication unit 130. Further, the processing unit 110 acquires information being a master of the authentication from the storage unit 150.

Note that, authentication processing of the target person may be performed by using identification information about the target person, for example, an ID, or may be performed by using biometric information other than face information, for example, at least one of iris information, fingerprint information, and vein information. Also, in this case, the processing unit 110 acquires information needed for authentication from the second terminal 30 via the communication unit 130.

The authentication unit 110 may dynamically switch criteria for the authentication processing. An example of a method for switching the criteria for the authentication processing will be described below.

For example, a stricter criterion for authentication has the advantage of increasing accuracy of the authentication of the target person. However, requirement for the criterion for the authentication that is excessively strict may result in a heavy load on the authentication processing and a time-consuming authentication processing.

Therefore, the authentication unit 110 may switch the criteria for the authentication processing by using existing machine learning techniques or optimization methods. As one example, the authentication unit 110 may use a machine learning model that receives as input information relating to a time and frequency at which the authentication processing was performed for the target person and determines the conditions required for the authentication process for the target person. For example, the input information is historical data relating to authentication processing performed for each target person and stored in an authentication result information storage unit, not shown.

For example, the machine learning model determines that the criteria for authentication processing will be relaxed, for a target person whose frequency of performing authentication processing is higher than a predetermined threshold or for a target person for whom the number of performing authentication processing within a predetermined period is equal to or more than a predetermined number. For a target person whose frequency of successful authentications is higher than the predetermined threshold or for a target person for whom the number of performing authentication processing within the predetermined period is equal to or more than the predetermined number, the machine learning model may determine that a frequency of requesting authentication should be reduced. On the other hand, for a target person whose frequency of performing authentication processing is not higher than the predetermined threshold or for a target person for whom the number of performing authentication processing within the predetermined period is less than the predetermined number of times, the machine learning model determine to tighten the criteria for authentication processing.

The authentication unit 110 can search for (or optimize) the appropriate first condition by using such a machine learning model.

In this way, by using the machine learning model created from historical data relating to the authentication processing, the authentication unit 230 can set a criterion for the authentication processing that can reduce a load of the authentication processing and a burden on the target person as much as possible, while maintaining authentication accuracy.

Further, the processing unit 110 generates state information by processing an image in which the target person is captured. Hereinafter, the image used when the state information is generated will be described as a first image. The first image may be an image used for authentication processing of the target person, or may be an image other than the image. Note that, a specific example of the state information will be described in a usage example of the communication apparatus 10 described below.

The judgement unit 120 performs, by using the state information, at least one of processing of determining first information needed to be transmitted to the first terminal 20 and processing of deciding whether to transmit the first information to the first terminal 20. Details of the processing performed herein will be described in the usage example of the communication apparatus 10 described below.

The communication unit 130 transmits and receives information to and from the first terminal 20, and also transmits and receives information to and from the second terminal 30. A specific example of the information transmitted and received by the communication unit 130 will be described in the usage example of the communication apparatus 10 described below.

The determination unit 140 determines, for the communication unit 130, whether transmission/reception of information to and from the first terminal 20 is enabled. For example, when a predetermined condition is not satisfied, the determination unit 140 does not permit the communication unit 130 to communicate with the first terminal 20. In other words, when the predetermined condition is not satisfied, the communication unit 130 does not perform communication with the first terminal 20. For example, when the target person is a resident in a facility, the predetermined condition is that there is a predetermined input from an assistant of the resident. For example, the predetermined input indicates that contact with a relative is permitted.

The storage unit 150 stores, by target person, information about the target person. One example of the information is information being a master of authentication.

Note that, in a case of the example illustrated in FIG. 1, the communication apparatus 10 is preferably a portable terminal such as a smartphone and a tablet type terminal. Further, the communication apparatus 10 also further has functions the same as the second terminal 30 as described above.

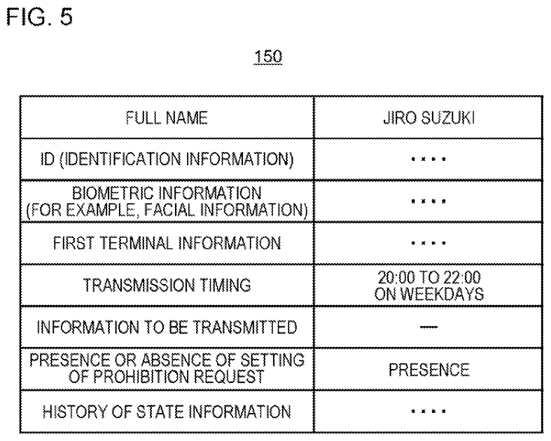

FIG. 5 is a diagram illustrating one example of information stored in the storage unit 150. The storage unit 150 stores, by target person, a name, identification information, biometric information being a master, first terminal information, transmission timing information, information to be transmitted, presence or absence of setting of a prohibition request, and a history of state information.

The identification information and the biometric information being the master are used when the processing unit 110 performs authentication processing. The storage unit 150 may not store one of the identification information and the biometric information. Note that, the storage unit 150 may store a password together with the identification information. In this case, the processing unit 110 authenticates a target person by using a combination of the identification information and the password.

The first terminal information stores information needed when the communication unit 130 is connected to the first terminal 20 associated with the target person. The first terminal information is, for example, at least one of a phone number, an e-mail address, and an account of an SNS (Social Networking Service).

The transmission timing information indicates a timing at which there is a high possibility that a user of the first terminal 20 operates the first terminal 20. In other words, the transmission timing information indicates a timing at which information is preferably transmitted from the communication apparatus 10 to the first terminal 20. This timing is indicated by, for example, a time period by day of a week, which is not limited thereto.

The transmission timing information may be set by a manual input from the target person, an assistant, or the user of the first terminal 20, or may be set based on an operation history of the first terminal 20. In the latter case, for example, the determination unit 140 of the communication apparatus 10 acquires, from the first terminal 20, an access history to information transmitted from the communication unit 130, aggregates, by day of a week, a time period in which an access frequency is high in this access history, and sets the transmission timing information by using this aggregation result. Note that, the determination unit 140 may set the transmission timing information by using an access history to an SNS by the first terminal 20.

Note that, the transmission timing information may not be set.

The “information to be transmitted” in the storage unit 150 in FIG. 5 is an area of a field (one of items in a record) in which information needed to be transmitted to the first terminal 20 is stored. The communication unit 120 acquires, from the second terminal 30, for example, the information to be transmitted to the first terminal 20, but may not immediately transmit the information to the first terminal 20. One example of this case is, for example, a case where the transmission timing information described above is set. In such a case, the communication unit stores the information acquired from the second terminal 30 in the area of the field “information to be transmitted” in the storage unit 150. Note that, the information to be transmitted may be deleted or left as it is after the information is transmitted to the first terminal 20.

The presence or absence of setting of a prohibition request indicates whether to allow a person other than a target person, for example, an assistant to recognize reply information being information to be output from the second terminal 30 when the reply information is received from the first terminal 20. When there is the setting, the communication unit 130 transmits, to the second terminal 30, the reply information together with prohibition request information indicating that the information should not be recognized by a person other than the target person.

Note that, the presence or absence of setting of a prohibition request may be set by kind of information. The kind of information is, for example, text data, an image, and a sound, which is not limited thereto. For example, setting that viewing by a person other than a target person is not prohibited for text data, but viewing by a person other than the target person is prohibited in a case of an image can be achieved.

The history of state information is information in which state information generated by the processing unit 110 is associated with a generation date and time of a first image used when the state information is generated. In other words, this history indicates a transition of past state information. This history may further include the first image.

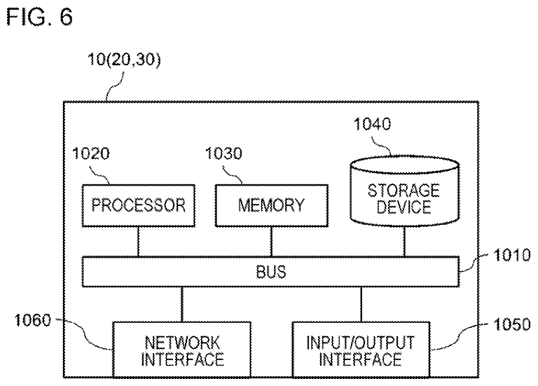

FIG. 6 is a diagram illustrating a hardware configuration example of the communication apparatus 10. The communication apparatus 10 includes a bus 1010, a processor 1020, a memory 1030, a storage device 1040, an input/output interface 1050, and a network interface 1060.

The bus 1010 is a data transmission path for allowing the processor 1020, the memory 1030, the storage device 1040, the input/output interface 1050, and the network interface 1060 to transmit and receive data with one another. However, a method for connecting the processor 1020 and the like to one another is not limited to bus connection.

The processor 1020 is a processor implemented by a central processing unit (CPU), a graphics processing unit (GPU), and the like.

The memory 1030 is a main storage apparatus implemented by a random access memory (RAM) and the like.

The storage device 1040 is an auxiliary storage apparatus implemented by a hard disk drive (HDD), a solid state drive (SSD), a removable medium such as a memory card, a read only memory (ROM), or the like, and includes a storage medium. The storage medium of the storage device 1040 stores a program module that implements each function (for example, the processing unit 110, the judgement unit 120, the communication unit 130, and the determination unit 140) of the communication apparatus 10. The processor 1020 reads each program module onto the memory 1030 and executes the program module, and each function associated with the program module is implemented. The storage device 1040 may further function as the storage unit 150.

The storage medium storing the program module may include a non-transitory tangible medium usable by the computer 1000, and a program code readable by the computer 1000 (the processor 1020) may be embedded in the medium.

The input/output interface 1050 is an interface for connecting the communication apparatus 10 and various types of input/output equipment.

The network interface 1060 is an interface for connecting the communication apparatus 10 to a network. The network is, for example, a local area network (LAN) and a wide area network (WAN). A method of connection to the network by the network interface 1060 may be wireless connection or wired connection. For example, the communication apparatus 10 communicates with the first terminal 20 and the second terminal 30 via the network interface 1060.

Note that, a hardware configuration of the first terminal 20 and the second terminal 30 is also similar to the hardware configuration illustrated in FIG. 6. In this case, the storage medium of the storage device 1040 stores a program module that implements each function of the first terminal 20 and the second terminal 30.

The storage medium storing the program module may include a non-transitory tangible medium usable by the computer 1000, and a program code readable by the computer 1000 (the processor 1020) may be embedded in the medium.

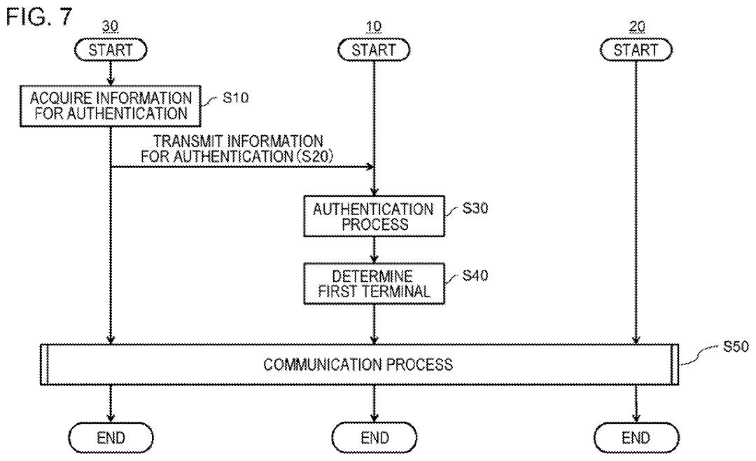

FIG. 7 is a flowchart illustrating one example of an operation performed by the communication apparatus 10. The example illustrated in FIG. 7 is associated with the example illustrated in FIG. 2.

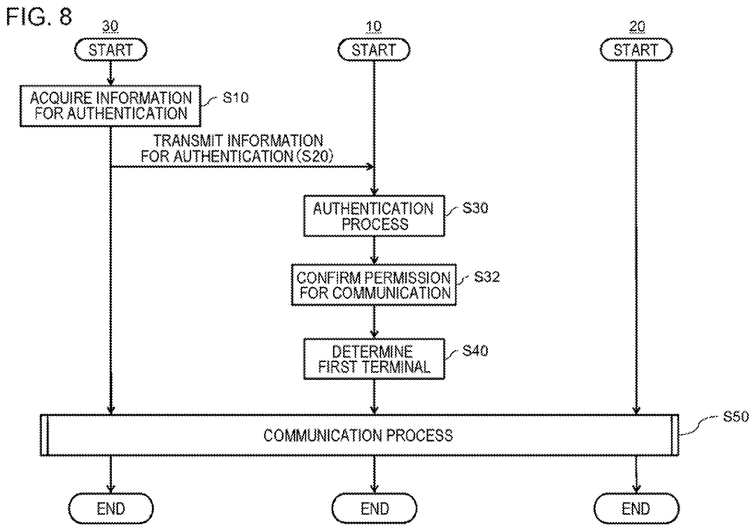

However, in the case of the example illustrated in FIG. 1, the communication apparatus 10 also performs processing indicated in step S10 in FIG. 7. Further, processing indicated in step S20 is not performed. This is also similar in an example illustrated in FIG. 8 described below.

When communication needs to be performed between a target person or an assistant, and a user of the first terminal 20, the second terminal 30 first acquires information needed for authenticating the target person, i.e., authentication information. For example, when the authentication information is face information, the input unit 310 of the second terminal 30 generates an image including a face of the target person. This image may be a moving image, or may be a still image. On the other hand, when the authentication information is identification information such as an ID, the second terminal 30 acquires the identification information via an input apparatus such as a touch panel (step S10).

Then, the communication unit 320 of the second terminal 30 transmits the authentication information to the communication apparatus 10 (step S20). The communication unit 130 of the communication apparatus 10 acquires the authentication information. Next, the processing unit 110 of the communication apparatus 10 authenticates the target person by using the authentication information, and information being stored in the storage unit 150. At this time, the target person is also determined (step S30).

Next, the communication unit 130 of the communication apparatus 10 reads, from the storage unit 150, first terminal information associated with the target person determined in step S30. In this way, the first terminal 20 being a communication partner this time is determined (step S40).

Subsequently, the communication apparatus 10 transmits and receives information to and from the first terminal 20, and also transmits and receives information to and from the second terminal 30. The transmitted and received information includes at least one of a sound, an image, and text data. In this way, the first terminal 20 and the second terminal 30 can transmit and receive the information via the communication apparatus 10. Note that, a specific example of the processing performed herein will be described below by using another diagram (step S50).

Note that, in step S20, the communication unit 320 of the second terminal 30 may transmit, to the communication apparatus 10, information needed to be transmitted to the first terminal 20 together with the authentication information. One example of the information transmitted herein is a message needed to be notified from the target person to the user of the first terminal 20. This message may be a video message, only a sound, or text data. Further, when the authentication information is an image, the authentication information may also function as a message. Such an example is suitable when this image is a moving image.

FIG. 8 is a diagram illustrating a modification example of FIG. 7. In the example illustrated in FIG. 8, processing until step S30 is similar to the processing in the example illustrated in FIG. 7. After authentication of a target person by the processing unit 110 of the communication apparatus 10 has succeeded (step S30), the determination unit 140 of the communication apparatus 10 confirms that communication with the first terminal 20 is permitted (step S32).

For example, a case where a target person is a resident in a facility, and the communication apparatus 10 is operated by an assistant of the resident is considered. In this case, when information indicating permission for communication is input from the assistant, the determination unit 140 decides that the communication with the first terminal 20 is permitted. Note that, in the present example, the communication apparatus 10 may be a portable terminal, a desktop personal computer, or a notebook computer. Further, the input of the information by the assistant may be performed via a terminal different from the communication apparatus 10.

Subsequent processing (steps S40 and S50) is as described by using FIG. 7. Note that, in step S32, when it cannot be confirmed that the communication with the first terminal 20 is permitted, the processing indicated in steps S40 and S50 is not performed.

### (First Specific Example of Step S50)

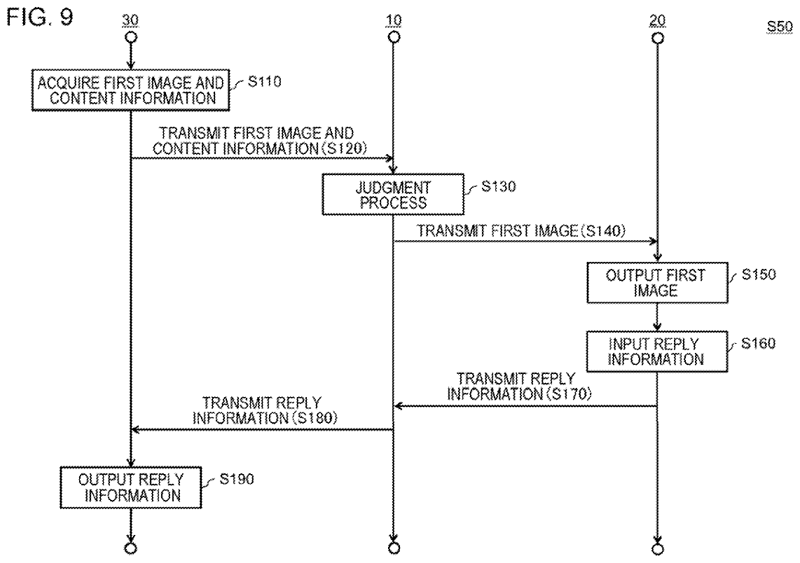

FIG. 9 is a flowchart illustrating a first specific example of step S50. The example illustrated in FIG. 9 illustrates a case where information about a target person is transmitted to a user of the first terminal 20. One example of the target person is a resident in a facility. In this case, the user of the first terminal 20 is a relative of the target person. Conversely, the target person may be a relative of a resident in a facility, and the user of the first terminal 20 may be the resident in the facility. An image is preferably a moving image, but may be a still image. Further, text data may be used instead of the image. Note that, the second terminal 30 may be operated by the resident, or may be operated by an assistant.

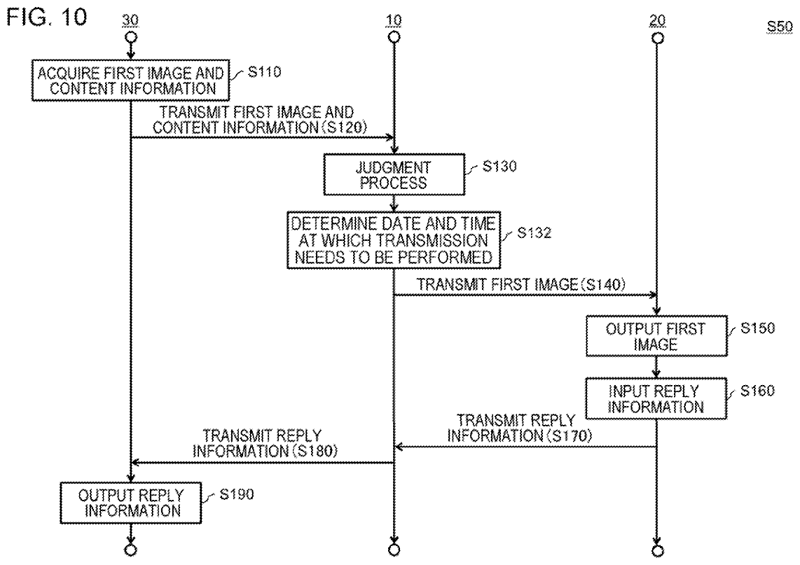

The example illustrated in FIG. 9 is associated with the example illustrated in FIG. 2. However, in the case of the example illustrated in FIG. 1, the communication apparatus 10 also performs processing indicated in steps S110 and S190 in FIG. 9. Further, processing indicated in steps S120 and S180 is not performed. This is also similar in an example illustrated in FIG. 10 described below.

First, the target person or the assistant causes the input unit 310 of the second terminal 30 to generate an image in which the target person is captured, i.e., a first image. The first image may be information needed to be transmitted to the first terminal 20, or may be information not transmitted to the first terminal 20. In the example illustrated in FIG. 9, the first image is information needed to be transmitted to the first terminal 20. The first image may be a message from the target person to the user of the first terminal 20, or may be an image in which a daily operation of the target person is captured (step S110). Note that, the input unit 310 of the second terminal 30 may generate sound data together with the first image.

The first image is generated while the target person is viewing a content or after the target person views the content, for example.

Herein, the content may be an image in which a relative of the target person, for example, the user of the first terminal 20 is captured. One example of this image is a moving image, for example, a video message from this relative to the target person, but may be a still image. In this case, the content is output from, for example, the second terminal 30. This content may be transmitted from the first terminal 20 to the second terminal 30 via the communication apparatus 10. In this case, this content is generated by, for example, the first terminal 20.

Further, the content may be a paper medium, for example, a magazine and a book, or may be output to a display different from the first terminal 20. An example of the latter is a television program.

Further, in step S110, the second terminal 30 acquires information indicating a kind of the content viewed by the target person when the first image is generated. Hereinafter, the information will be described as content information. When the content is a video message from a relative, the content information includes a name of the relative, and a generation date and time or a transmission date and time of the video message. When the content is a paper medium, the content information includes a name of this paper medium, for example, a name of a book and a magazine. When the content is a television program, the content information includes a name of the program, and a broadcast date and time. The content information may be input to the second terminal 30 by a manual input, may be generated by processing the first image, or may be generated by another method. Note that, the content information may include the content itself.

Note that, the first image may be generated by a capturing apparatus other than the second terminal 30, for example, a surveillance camera installed in the facility.

This capturing apparatus may directly transmit the generated image to the communication apparatus 10. In this case, for example, after step S40 in FIG. 7, the communication apparatus 10 requests an image from this capturing apparatus. Then, this capturing apparatus transmits the image to the communication apparatus 10.

Further, this capturing apparatus may transmit the generated image to the second terminal 30. In this case, the second terminal 30 transmits, to the communication apparatus 10, the image received from the capturing apparatus. However, this capturing apparatus may directly transmit the image to the first terminal 20.

Next, the communication unit 320 of the second terminal 30 transmits the first image and the content information to the communication apparatus 10. When the input unit 310 further generates sound data, the communication unit 320 also further transmits the sound data to the communication apparatus 10 (step S120).

Note that, the first image may be an image used when the target person is authenticated. In this case, the processing indicated in steps S110 and S120 may be not performed. When those steps are not performed, acquisition and transmission of the content information are performed in steps S10 and S20 in FIG. 8.

The communication unit 130 of the communication apparatus 10 acquires the first image and the content information. Then, the processing unit 110 of the communication apparatus 10 generates state information by processing the first image. When the second terminal 30 also transmits the sound data, the processing unit 110 may further use the sound data when the state information is generated. The state information indicates a state of the target person. One example of the state of the target person includes at least one of a feeling and a physical condition of the target person when the first image is generated. Then, the processing unit 110 stores the generated state information in association with a generation date and time of the first image in the storage unit 150.

Then, the judgement unit 120 performs, by using the state information generated by the processing unit 110, at least one of processing of determining first information needed to be transmitted to the first terminal 20 and processing of deciding whether to transmit the first information to the first terminal 20 (step S130).

For example, when the state information indicates that the target person is glad, the judgement unit 120 decides that the first information needs to be transmitted. Further, when the state information indicates that the target person is glad, the judgement unit 120 includes at least one of the first image, the state information, and the content information in the first information. The first information may be any one of the three, may be any two of the three, or may include all of the three.

For example, in a case where the first image is a video indicating a fact that the target person is glad, for example, a relative of the target person can directly confirm this fact when the first image is included in the first information. Further, when information about a fact that the “target person seems glad” being one example of the state information is included in the first information, for example, a relative of the target person can recognize this fact.

Conversely, in a case where the first image is a video indicating a fact that a feeling of the target person is negative (sad, angry, depressed, or the like), for example, a relative of the target person can directly confirm this fact when the first image is included in the first information. Further, when information about a fact that the “target person has a negative feeling” being one example of the state information is included in the first information, for example, a relative of the target person can recognize this fact.

Note that, the user of the communication apparatus 10 may be able to previously set which of the first image, the state information, and the content information needs to be included in the first information. In this case, the judgement unit 120 of the communication apparatus 10 determines information needed to be included in the first information according to the setting.

Note that, when the state information indicates that the target person has a negative feeling, the judgement unit 120 may decide that the first information is not to be transmitted.

Next, the communication unit 130 of the communication apparatus 10 transmits the first information to the first terminal 20 (step S140).

The communication unit 220 of the first terminal 20 acquires the first information from the communication apparatus 10. Then, the output unit 230 of the first terminal 20 outputs the first information (step S150). In this way, the user of the first terminal 20 can recognize the first information.

Then, the user of the first terminal 20 inputs, to the first terminal 20, information to be transmitted to the target person. Hereinafter, the information will be described as reply information. For example, the first terminal 20 generates a second image as at least a part of the reply information by using the input unit 210. The second image may be an image in which the user viewing the first image is captured, or may be an image in which the user after viewing the first image is captured. This example is suitable when the second image is a message from a relative of the resident in the facility to the resident, for example (step S160).

Then, the communication unit 220 of the first terminal 20 transmits the reply information to the communication apparatus 10 (step S170). When the communication unit 130 of the communication apparatus 10 acquires the reply information from the first terminal 20, the communication unit 130 transmits the reply information to the second terminal 30 (step S180).

The communication unit 320 of the second terminal 30 acquires the reply information. Then, the output unit 330 of the second terminal 30 outputs the reply information, and causes the target person to recognize the reply information (step S190). Note that, when the reply information includes a moving image, and a reproduction time of the moving image is equal to or more than a reference value, the communication unit 320 may divide and output the moving image, or may fast-forward and output the moving image.

Note that, when the target person is a resident in a facility, and a medicine needs to be taken, a timing at which an output of the reply information and predetermined processing is performed, i.e., a timing at which step S190 is performed may be a timing at which the medicine is taken.

Further, the first information may include an image in which the target person is captured by a third terminal different from the second terminal 30. For example, a case where the second terminal 30 is operated by the target person, and the third terminal is operated by an assistant is assumed. In this case, the judgement unit 120 of the communication apparatus 10 transmits, to the third terminal via the communication unit 130, information that prompts capturing the target person between steps S130 and S140. Then, the third terminal outputs the information to, for example, a display. When the assistant recognizes the information, the assistant captures the target person, generates an image, and transmits the generated image to the communication apparatus 10. In step S140, after the communication unit 130 of the communication apparatus 10 includes this image as at least a part of the first information in the first information, the communication unit 130 transmits the first information to the first terminal 20.

Further, in step S130, the judgement unit 120 may perform, by using a history of the state information stored in the storage unit 150, at least one of processing of determining first information needed to be transmitted to the first terminal 20 and processing of deciding whether to transmit the first information to the first terminal 20.

For example, when a state where the state information about the target person satisfies a criterion continues for a fixed period, the judgement unit 120 includes information indicating the continuation as at least a part of the first information in the first information, and also decides that the first information needs to be transmitted. One example of a fact that the “state where the state information satisfies a criterion continues for a fixed period” is that indication of a negative feeling by the state information continues for a fixed time (for example, one week). In this case, it is desirable that a relative of the target person communicates with the target person. According to this example, the relative of the target person can easily recognize a timing at which communication with the target person needs to be performed.

Further, a case where the target person is in a care facility, a nursing home, or a hospital, and the first terminal 20 is used by a person in charge of the target person in the care facility, the nursing home, the hospital, or a pharmacy, such as at least one of a caregiver, a pharmacist, a nurse, and a doctor, is assumed. Then, the first image is assumed to include an image when the target person takes a medicine. Herein, the “image when medicine is given by hand” is at least one of an image when the target person is given the medicine, an image when the target person swallow the medicine, and an image immediately after the target person swallow the medicine. The first image may be a still image, or may be a moving image. In this case, when the number of times the state information satisfies a criterion is equal to or more than a predetermined number, the judgement unit 120 may include, as at least a part of the first information in the first information, at least one of information indicating that the number of times is equal to or more than the predetermined number and information indicating a kind of a medicine prescribed to the target person. One example of the criterion used herein is indication of a negative feeling by the state information. In this case, there is a possibility that the target person refuses a currently prescribed medicine. According to this example, the pharmacist can easily recognize this possibility.

Note that, the first image may be generated by capturing the target person at a timing at which an effect of a medicine is expected to be acquired. In this case, the state information indicates an estimation result of whether a medicine being previously taken is effective. Further, the criterion used by the judgement unit 120 relates to this estimation result.

For example, when an expression, a movement, and a pose of the target person are good, and there are less disheveled clothing and disheveled hair of the target person, the judgement unit 120 decides that a medicine being previously taken by the target person is effective. On the other hand, when an expression, a movement, and a pose of the target person are bad, and there are more disheveled clothing and disheveled hair of the target person, the judgement unit 120 decides that a medicine being previously given is unsuitable for the target person. Further, when the first image includes an image before a medicine this time is given to the target person, the judgement unit 120 may perform the decision described above by processing this image.

In this case, the processing unit 110 may generate status information indicating an estimated result of whether the previously taken medication was effective or not, using a machine learning model that has learned correspondence between an image when the target takes the medication and information indicating whether or not the medication is appropriate for the target person.

Then, when this decision result continues for a reference number of times, the judgement unit 120 includes information indicating this decision result as at least a part of the first information in the first information, and decides that the first information needs to be transmitted to the first terminal 20. Note that, the reference number of times described above may be plural or one. According to this example, a pharmacist can easily recognize an effect of a medicine being prescribed.

Specifically, if a result indicating that a previously taken medication is not effective as an estimated result indicated by the status information occurs a criterion number of consecutive times, the judgement unit 120 decides that information indicating the result of this decision should be included as at least part of the first information in the first information and this first information should be transmitted to the first terminal 20.

Furthermore, physicians and pharmacists can optimize the medications they prescribe to the target person based on the first information.

The judgement unit 120 may also use the machine learning model to generate an explanatory text for the first image of the target person and include it as part of the first information in the first information. For example, the judgement unit 120 acquires the first image of the target person, analyzes detailed features of the first image using the machine learning model, and generates the explanatory text that “The hair of the target person is less disheveled, and his posture is well adjusted. He is also active in his movements.” and so on. The judgment unit 120 generates such explanatory text, includes it as a part of the first information in the first information, and cause the pharmacist and other healthcare professionals to read this explanatory text, this results in that the pharmacist and other healthcare professionals can be assisted in decision making.

A method for generating explanatory text from images using a machine learning model includes methods by using, for example, Neural Image Caption (NIC) and Transformer models. NIC extracts visual feature from input image by using a machine learning model including a combination of Convolutional Neural Networks (CNN) and Recurrent Neural Networks (RNN) and generate an explanatory text based on the extracted visual feature.

Transformer model is able to generate an explanatory text based on how different parts of the input image interact with each other using a technique called a self-attention mechanism.

### (Second Specific Example of Step S50)

FIG. 10 is a flowchart illustrating a second specific example of step S50. The example illustrated in FIG. 10 is similar to the specific example illustrated in FIG. 9 except for a point that a timing at which the communication apparatus 10 transmits first information to the first terminal 20 is not in real time, and is preset by target person.

In FIG. 10, processing until step S130 is similar to the processing in the example illustrated in FIG. 9. After step S130, the communication unit 130 of the communication apparatus 10 reads, from the storage unit 150, a transmission timing associated with a target person determined in step S30 in FIG. 7. In this way, the communication unit 130 determines a date and time at which the first information needs to be transmitted (step S132). Then, the communication unit 130 transmits the first information to the first terminal 20 on the determined date and time (step S140).

Subsequent processing (steps S150 to S190) is similar to the processing in the example illustrated in FIG. 9. Note that, also in another example described below, similarly to the present example, the processing indicated in steps S132 and S140 may be performed.

### (Third Specific Example of Step S50)

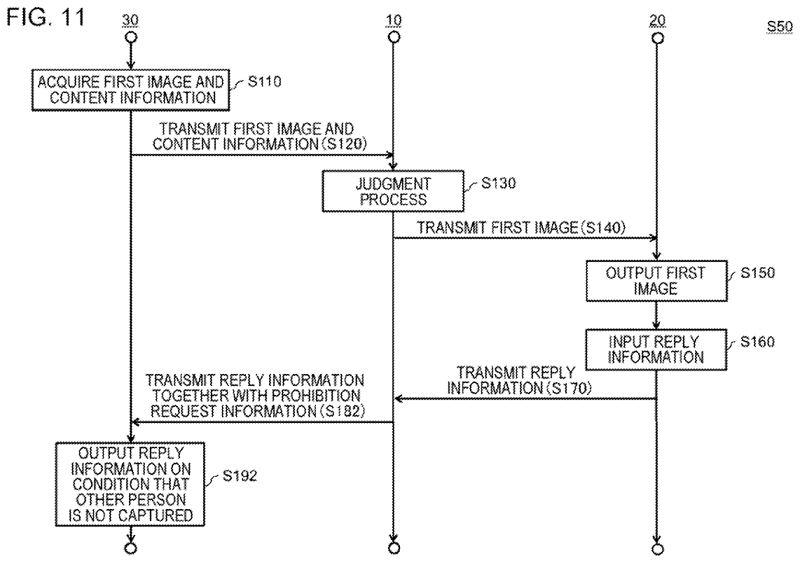

FIG. 11 is a flowchart illustrating a third specific example of step S50. The example illustrated in FIG. 11 is similar to the specific example illustrated in FIG. 9 except for a point that the communication apparatus 10 transmits prohibition request information together with reply information, and a point that the output unit 330 of the second terminal 30 operates based on the prohibition request information. The prohibition request information indicates that a person other than a target person is prohibited from recognizing the reply information.

The example illustrated in FIG. 11 is associated with the example illustrated in FIG. 2. However, in the case of the example illustrated in FIG. 1, the communication apparatus 10 also performs processing indicated in steps S110 and S192 in FIG. 11. Further, processing indicated in steps S120 and S182 is not performed.

In FIG. 11, processing until step S170 is similar to the processing in the example illustrated in FIG. 9. When the communication unit 130 of the communication apparatus 10 acquires the reply information from the first terminal 20, the communication unit 130 confirms, from the storage unit 150, whether a prohibition request is set for a target person determined in step S30 in FIG. 7. In other words, the communication unit 130 decides whether it is necessary to prohibit a person other than a resident from recognizing the reply information, based on the target person. When there is no setting of the prohibition request, processing similar to the processing in FIG. 9 is subsequently performed. On the other hand, when there is the setting of the prohibition request, the communication unit 130 performs processing for prohibiting a person other than the resident from recognizing the reply information. In the example illustrated in FIG. 11, the communication unit 130 transmits the reply information together with the prohibition request information to the second terminal 30 (step S182). It means that, in the processing, the communication unit 130 decides whether transmission of the prohibition request information is necessary, based on the target person.

Subsequently, the second terminal 30 causes the output unit 330 to output the reply information while performing capturing by the input unit 310. Herein, the output unit 330 outputs the reply information only while a face of the target person is detected from an image captured by the input unit 310. Further, when a person other than the target person is detected from the image captured by the input unit 310, the output unit 330 does not perform an output of the reply information. Further, when a person other than the target person is detected from the image captured by the input unit 310 during an output of the reply information, the output of the reply information is interrupted, and then, when only the target person is detected from the image captured by the input unit 310, the output of the reply information is restarted (step S192).

Note that, in the example illustrated in FIG. 11, processing of an image captured by the input unit 310 is performed by the second terminal 30. However, the processing may be performed by the communication apparatus 10. In this case, every time the input unit 310 generates an image, for example, a frame image, the input unit 310 transmits the image to the communication apparatus 10. Then, the processing unit 110 of the communication apparatus 10 decides whether an image of the target person is being detected from the acquired image, and whether a person other than the target person is being detected from this image, and transmits a decision result to the second terminal 30. Then, the output unit 330 of the second terminal 30 controls the output of the reply information by using the decision result acquired from the communication apparatus 10.

As described above, the present example embodiment can use a state of a person when communication between a plurality of persons is supported. Specifically, the judgement unit 120 of the communication apparatus 10 performs, by using state information indicating a state of a target person, at least one of processing of determining first information needed to be transmitted to the first terminal 20 and processing of deciding whether to transmit the first information to the first terminal 20. Thus, information can be transmitted to the first terminal 20 at an appropriate timing, and information about an appropriate content can be transmitted to the first terminal 20.

While the example embodiments of the present invention have been described with reference to the drawings, the example embodiments are only exemplification of the present invention, and various configurations other than the above-described example embodiments can also be employed.

Further, the plurality of steps (pieces of processing) is described in order in the plurality of flowcharts used in the above-described description, but an execution order of steps performed in each of the example embodiments is not limited to the described order. In each of the example embodiments, an order of illustrated steps may be changed within an extent that there is no harm in context. Further, each of the example embodiments described above can be combined within an extent that a content is not inconsistent.

While the invention has been particularly shown and described with reference to example embodiments thereof, the invention is not limited to these embodiments. It will be understood by those of ordinary skill in the art that various changes in form and details may be made therein without departing from the spirit and scope of the present invention as defined by the claims.

The whole or part of the example embodiments disclosed above can be described as, but not limited to, the following supplementary notes.

1. A communication apparatus including:
a processing unit that generates state information indicating a state of a target person by processing an image in which the target person is captured;
a judgement unit that performs, by using the state information, at least one of processing of determining first information needed to be transmitted to a first terminal being associated with the target person and being operated by a different person from the target person and processing of deciding whether to transmit the first information to the first terminal; and
a communication unit that transmits the first information to the first terminal.
   2. The communication apparatus according to supplementary note 1, wherein
the image is generated while the target person is viewing a content or after the target person views the content.
   3. The communication apparatus according to supplementary note 2, wherein
the content includes an image in which a relative of the target person is captured.
   4. The communication apparatus according to supplementary note 2 or 3, wherein
the judgement unit includes the image in the first information when the state information indicates that the target person is glad.
   5. The communication apparatus according to supplementary note 2, wherein
the judgement unit includes information indicating the content as at least a part of the first information in the first information when the state information indicates that the target person is glad.
   6. The communication apparatus according to any one of supplementary notes 1 to 5, wherein
the first terminal is used by a relative of the target person, and,
when a state where the state information satisfies a criterion continues for a fixed period, the judgement unit includes, as the first information in the first information, information indicating that the state continues for the fixed period as at least a part of the first information.
   7. The communication apparatus according to any one of supplementary notes 1 to 6, wherein
the target person is in a care facility, a nursing home, or a hospital,
the first terminal is used by a person in charge of the target person in the care facility, the nursing home, the hospital, or a pharmacy, and,
when a number of times the state information satisfies a criterion is equal to or more than a predetermined number, the judgement unit includes, as at least a part of the first information in the first information, at least one of information indicating that the number of times is equal to or more than the predetermined number and information indicating a kind of a medicine prescribed to the target person.
   8. A communication method including,
by at least one computer:
generating state information indicating a state of a target person by processing an image in which the target person is captured;
performing, by using the state information, at least one of processing of determining first information needed to be transmitted to a first terminal being associated with the target person and being operated by a different person from the target person and processing of deciding whether to transmit the first information to the first terminal; and
transmitting the first information to the first terminal.
   9. The communication method according to supplementary note 8, wherein
the image is generated while the target person is viewing a content or after the target person views the content.
   10. The communication method according to supplementary note 9, wherein
the content includes an image in which a relative of the target person is captured.
   11. The communication method according to supplementary note 9 or 10, further including,
by the computer, including the image in the first information when the state information indicates that the target person is glad.
   12. The communication method according to supplementary note 9, further including,
by the computer, including information indicating the content as at least a part of the first information in the first information when the state information indicates that the target person is glad.
   13. The communication method according to any one of supplementary notes 8 to 12, wherein
the first terminal is used by a relative of the target person,
the communication method further including,
by the computer, when a state where the state information satisfies a criterion continues for a fixed period, including, as the first information in the first information, information indicating that the state continues for the fixed period as at least a part of the first information.
   14. The communication method according to any one of supplementary notes 8 to 13, wherein
the target person is in a care facility, a nursing home, or a hospital, and
the first terminal is used by a person in charge of the target person in the care facility, the nursing home, the hospital, or a pharmacy,
the communication method further including,
by the computer, when a number of times the state information satisfies a criterion is equal to or more than a predetermined number, including, as at least a part of the first information in the first information, at least one of information indicating that the number of times is equal to or more than the predetermined number and information indicating a kind of a medicine prescribed to the target person.
   15. A program causing a computer to:
generate state information indicating a state of a target person by processing an image in which the target person is captured;
perform, by using the state information, at least one of processing of determining first information needed to be transmitted to a first terminal being associated with the target person and being operated by a different person from the target person and processing of deciding whether to transmit the first information to the first terminal; and
transmit the first information to the first terminal.
   16. The program according to supplementary note 15, wherein
the image is generated while the target person is viewing a content or after the target person views the content.
   17. The program according to supplementary note 16, wherein
the content includes an image in which a relative of the target person is captured.
   18. The program according to supplementary note 16 or 17, further causing the computer to
include the image in the first information when the state information indicates that the target person is glad.
   19. The program according to supplementary note 16, further causing the computer to
include information indicating the content as at least a part of the first information in the first information when the state information indicates that the target person is glad.
   20. The program according to any one of supplementary notes 15 to 19, wherein
the first terminal is used by a relative of the target person,
the program further causing the computer to, when a state where the state information satisfies a criterion continues for a fixed period, include, as the first information in the first information, information indicating that the state continues for the fixed period as at least a part of the first information.
   21. The program according to any one of supplementary notes 15 to 20, wherein
the target person is in a care facility, a nursing home, or a hospital, and
the first terminal is used by a person in charge of the target person in the care facility, the nursing home, the hospital, or a pharmacy,
the program further causing the computer to, when a number of times the state information satisfies a criterion is equal to or more than a predetermined number, include, as at least a part of the first information in the first information, at least one of information indicating that the number of times is equal to or more than the predetermined number and information indicating a kind of a medicine prescribed to the target person.
   22. A storage medium storing the program according to any one of supplementary notes 15 to 21.
   23. The communication apparatus according to supplementary note 7, wherein
the processing unit generates the status information indicating a result of estimating whether previously taken medicine is effective or not, using a machine learning model that has learned correspondence between an image when the target person takes the medicine and information indicating whether the medicine is suitable for the target person or not.
   24. The communication apparatus according to supplementary note 23, wherein
the criterion is that the state information is an estimated result indicating that the last time the medicine was taken was not effective.
   25. The communication apparatus according to supplementary note 7, wherein
the judgement unit generates an explanatory text for the image and includes the explanatory text as at least part of the first information in the first information.
   26. The communication method according to supplementary note 14, further including,
by the computer,
generating the status information indicating a result of estimating whether previously taken medicine is effective or not, using a machine learning model that has learned correspondence between an image when the target person takes the medicine and information indicating whether the medicine is suitable for the target person or not.
   27. The communication method according to supplementary note 26, wherein
the criterion is that the state information is an estimated result indicating that the last time the medicine was taken was not effective.
   28. The communication method according to supplementary note 14, further including,
by the computer,
generating an explanatory text for the image and including the explanatory text as at least part of the first information in the first information.
   29. The program according to supplementary note 21 further causing the computer to
generate the status information indicating a result of estimating whether previously taken medicine is effective or not, using a machine learning model that has learned correspondence between an image when the target person takes the medicine and information indicating whether the medicine is suitable for the target person or not.
   30. The program according to supplementary note 29, wherein
the criterion is that the state information is an estimated result indicating that the last time the medicine was taken was not effective.
   31. The program according to supplementary note 21 further causing the computer to generate an explanatory text for the image and include the explanatory text as at least part of the first information in the first information.
   32. A non-transitory computer-readable storage medium storing the program according to any one of supplementary notes 15 to 21 and 29 to 31.

What is claimed is:

1. A communication apparatus comprising:
at least one memory configured to store instructions; and
at least one processor configured to execute the instructions to:
generate state information indicating a state of a target person by processing an image in which the target person is captured;
perform, by using the state information, at least one of processing of determining first information needed to be transmitted to a first terminal being associated with the target person and being operated by a different person from the target person and processing of deciding whether to transmit the first information to the first terminal; and
transmit the first information to the first terminal.

2. The communication apparatus according to claim 1, wherein
the image is generated while the target person is viewing a content or after the target person views the content.

3. The communication apparatus according to claim 2, wherein
the content includes an image in which a relative of the target person is captured.

4. The communication apparatus according to claim 2, wherein
the at least one processor is further configured to execute the instructions to include the image in the first information when the state information indicates that the target person is glad.

5. The communication apparatus according to claim 2, wherein
the at least one processor is further configured to execute the instructions to include information indicating the content as at least a part of the first information in the first information when the state information indicates that the target person is glad.

6. The communication apparatus according to claim 1, wherein
the first terminal is used by a relative of the target person, and
the at least one processor is further configured to execute the instructions to, when a state where the state information satisfies a criterion continues for a fixed period, include information indicating that the state continues for the fixed period as at least a part of the first information in the first information.

7. The communication apparatus according to claim 1, wherein
the target person is in a care facility, a nursing home, or a hospital,
the first terminal is used by a person in charge of the target person in the care facility, the nursing home, the hospital, or a pharmacy, and
the at least one processor is further configured to execute the instructions to, when a number of times the state information satisfies a criterion is equal to or more than a predetermined number, include, as at least a part of the first information in the first information, at least one of information indicating that the number of times is equal to or more than the predetermined number and information indicating a kind of a medicine prescribed to the target person.

8. The communication apparatus according to claim 7, wherein
the at least one processor is further configured to execute the instructions to generate the status information indicating a result of estimating whether previously taken medicine is effective or not, using a machine learning model that has learned correspondence between an image when the target person takes the medicine and information indicating whether the medicine is suitable for the target person or not.

9. The communication apparatus according to claim 8, wherein
the criterion is that the state information is an estimated result indicating that the last time the medicine was taken was not effective.

10. The communication apparatus according to claim 7, wherein
the at least one processor is further configured to execute the instructions to generate an explanatory text for the image and include the explanatory text as at least part of the first information in the first information.

11. A communication method comprising,
by at least one computer:
generating state information indicating a state of a target person by processing an image in which the target person is captured;
performing, by using the state information, at least one of processing of determining first information needed to be transmitted to a first terminal being associated with the target person and being operated by a different person from the target person and processing of deciding whether to transmit the first information to the first terminal; and
transmitting the first information to the first terminal.

12. A non-transitory computer-readable storage medium storing a program causing a computer to execute:
a process of generating state information indicating a state of a target person by processing an image in which the target person is captured;
a process of performing, by using the state information, at least one of processing of determining first information needed to be transmitted to a first terminal being associated with the target person and being operated by a different person from the target person and processing of deciding whether to transmit the first information to the first terminal; and
a process of transmitting the first information to the first terminal.
